(19) United States
(12) Patent Application Publication
(10) Pub. No.: US 2007/0005065 A1
(43) Pub. Date: Jan. 4, 2007

(54) Aiming arm hole shaped to perform an incision through, and method to use that same

(76) Inventor: Alberto Angel Fernandez Dell'Oca, Montevideo (UY)

Correspondence Address: JONES DAY, 222 EAST 41ST STREET, NEW YORK, NY, 10017-6702, US

(21) Appl. No.: 11/154,653

(22) Filed: Jun. 17, 2005

(51) Int. Cl. A61F 2/30

(52) U.S. Cl. 606/62

(57) ABSTRACT

A specially shaped hole in an aiming device which enables the surgeon to perform an incision through the aiming device transverse hole in a plane that contains nail hole axis. Incision is therefore important to prevent undesirable side pull stress over the drill guide inserted through the aiming device transverse hole, which causes apparent problems in order to fit the drill through the hole in the nail. Thus, at least one screw for interlocking the nail with surrounding bone can accurately be installed.

### CROSS-REFERENCE TO RELATED APPLICATIONS

U.S. Patent Document

| | | |
|---|---|---|
| 4667664 | May 1987 | Taylor et al. |
| 4865025 | September 1989 | Buzzi et al. |
| 4976713 | December 1990 | Landanger et al. |
| 5334192 | August 1994 | Behrens |
| 5766179 | June 1998 | Faccioli et al. |
| 6074394 | June 2000 | Krause |
| 6214013 | April 2001 | Lambrecht et al. |
| 6869434 | March 2005 | Choi |

STATEMENT REGARDING FEDERALLY SPONSORED RESEARCH OR DEVELOPMENT

Not applicable.

REFERENCE TO SEQUENCE LISTING, A TABLE, OR A COMPUTER PROGRAM LISTING COMPACT DISC APPENDIX

Not applicable.

### BACKGROUND OF THE INVENTION

The present invention relates to a specially shaped hole in an aiming device so that a cutting instrument can be inserted through it in order to perform an incision.

To better understand the problem faced by the inventor and resolved by the invention, reference will first be made to the prior art shown in FIGS. 1A, 1B, 1C, 2A, 2B and 2C.

Locking nails 10 insertable into long bones are usually provided with transverse holes 11. Screws can be inserted through the holes 11 for interlocking the nail 10 with the surrounding bone B. A special problem with locking nails 10 is one of assuring correct alignment of drill with the transverse hole 11 of the implanted nail 10. Aiming devices 20 are often used for this purpose.

Currently available aiming devices transverse holes 26 do not enable the surgeon to insert a cutting instrument C through them in order to perform an incision I.

As shown in the cross-sectional diagram of FIG. 1B since the cutting instrument C can not go through aiming device transverse hole 26 the surgeon must perform the incision I by the side of the aiming device 0 in a plane that does not contain aiming device transverse hole 26 axis. Thus, once the guiding sleeve 30 is inserted through the transverse hole 26 of the aiming device 20 and slides through soft tissues until the external surface of the bone B is reached, an undesirable side pulling stress 18 appears over such sleeves 30 (see FIG. 1C).

Since this pull 18 may move the guiding sleeve 30 from the correct position (see FIG. 1D), the hole drilled through the bone B through which the screw is inserted, may not be aligned co-axially with the nail hole 11. If the guide hole 31 is not co-axial with the nail hole 11 axis, the anchoring screw will not perfectly fit through the hole 11 in the nail 10.

The invention aims to overcome the problems discussed above.

### SUMMARY OF THE INVENTION

The present invention has as one objective to enable the insertion of a scalpel through at least one of the transverse holes located in the aiming device while keeping the possibility to guide the drilling sleeve.

In one embodiment, a slot is combined with the existing aiming device transverse hole.

According to another embodiment, at least one additional hole is combined with the existing aiming device transverse hole.

According to a further embodiment of the present invention, an insert with a transverse hole is provided so that when fixed in the aiming device allows the drill guide to go through it. The insert when removed, enables the insertion of the scalpel through the aiming device.

The present invention also provides a method for performing an incision through the aiming device.

### BRIEF DESCRIPTION OF THE DRAWINGS

FIG. 1A is a cross sectional diagram showing positioning of the aiming device in a surgical procedure;

FIG. 1B is a cross sectional diagram showing the current process a surgeon uses to perform an incision when the aiming device is correctly positioned;

FIG. 1C is a cross sectional diagram showing the current process a surgeon uses to insert the drill guide aligned with nail hole axis while slides through soft tissues until the external surface of the bone is reached;

FIG. 1D is a cross sectional diagram showing how asymmetrical side pull stress moves the drill guide up and finishes to be misaligned from the nail hole axis;

FIG. 2A is a perspective view of the surgical process to perform an incision in order to insert the drill guide when the prior art aiming device is used;

FIG. 2B is a partially lateral view of the prior art aiming device illustrated in FIG. 2A;

FIG. 2C is a partially cut away and vertical cross-sectional view of the aiming device transverse hole illustrated in FIG. 2B taken along cut-away line 2C-2C in FIG. 2B;

FIG. 3A is a cross sectional diagram showing positioning of one preferred embodiment in a surgical procedure;

FIG. 3B is a cross sectional diagram showing the process a surgeon uses to perform an incision according to the present invention;

FIG. 3C is a cross sectional diagram showing the drill guide sliding through soft tissues while being aligned with nail hole axis, since asymmetrical side pull stress disappeared;

FIG. 4A shows in perspective a preferred embodiment of the aiming device shown in FIG. 2 with a special shaped hole comprising a round hole combined with a slot;

FIG. 4B is a partially lateral view of the aiming device illustrated in FIG. 4A;

FIG. 4C is a partially cut away and vertical cross-sectional view of the aiming device transverse hole illustrated in FIG. 4B taken along cut-away line 4C-4C in FIG. 4B;

FIG. 5A shows in perspective an alternate preferred embodiment of the aiming device shown in FIG. 2 with a special shaped hole comprising a round hole with two additional round holes one by each side;

FIG. 5B is a partially lateral view of the aiming device illustrated in FIG. 5A;

FIG. 5C is a partially cut away and vertical cross-sectional view of the aiming device transverse hole illustrated in FIG. 5B taken along cut-away line 5C-5C in FIG. 5B;

FIG. 6A shows in perspective another embodiment of the aiming device shown in FIG. 2 in which the aiming device transverse hole is situated in a removable insert;

FIG. 6B is a partially lateral view of the aiming device illustrated in FIG. 6A;

FIG. 6C is a partially cut away and vertical cross-sectional view of the aiming device transverse hole illustrated in FIG. 6B taken along cut-away line 6C-6C in FIG. 6B;

FIG. 6D is a partially cut away and vertical cross-sectional view of the aiming device illustrated in FIG. 6A showing the hole left in the aiming device when the insert is removed.

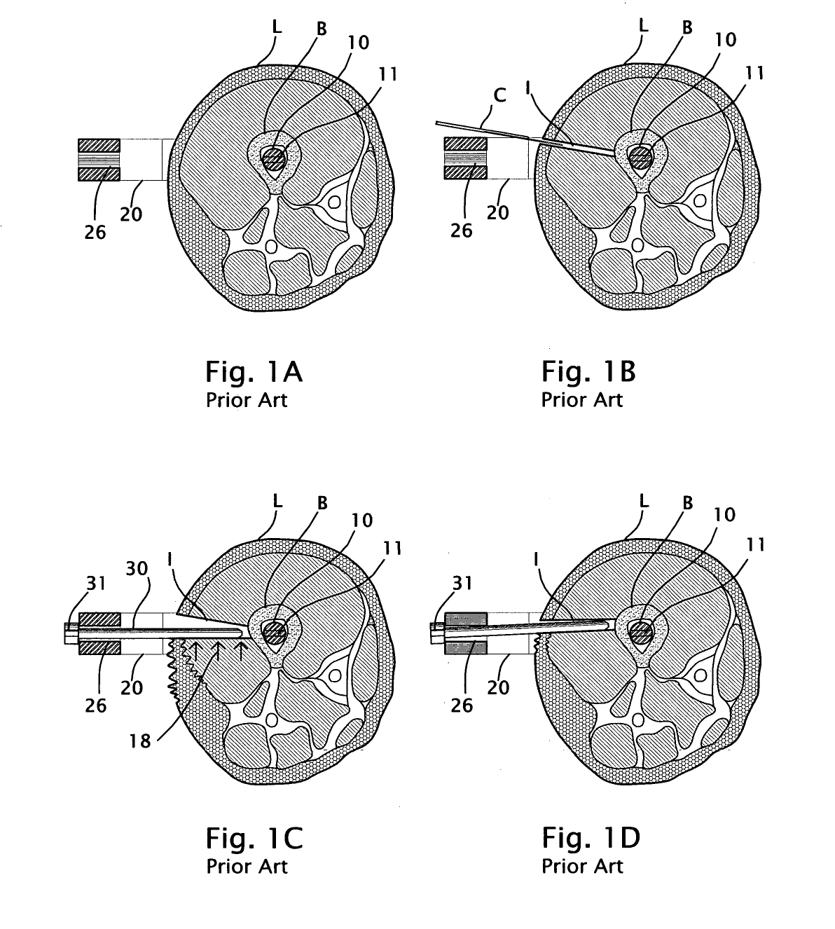

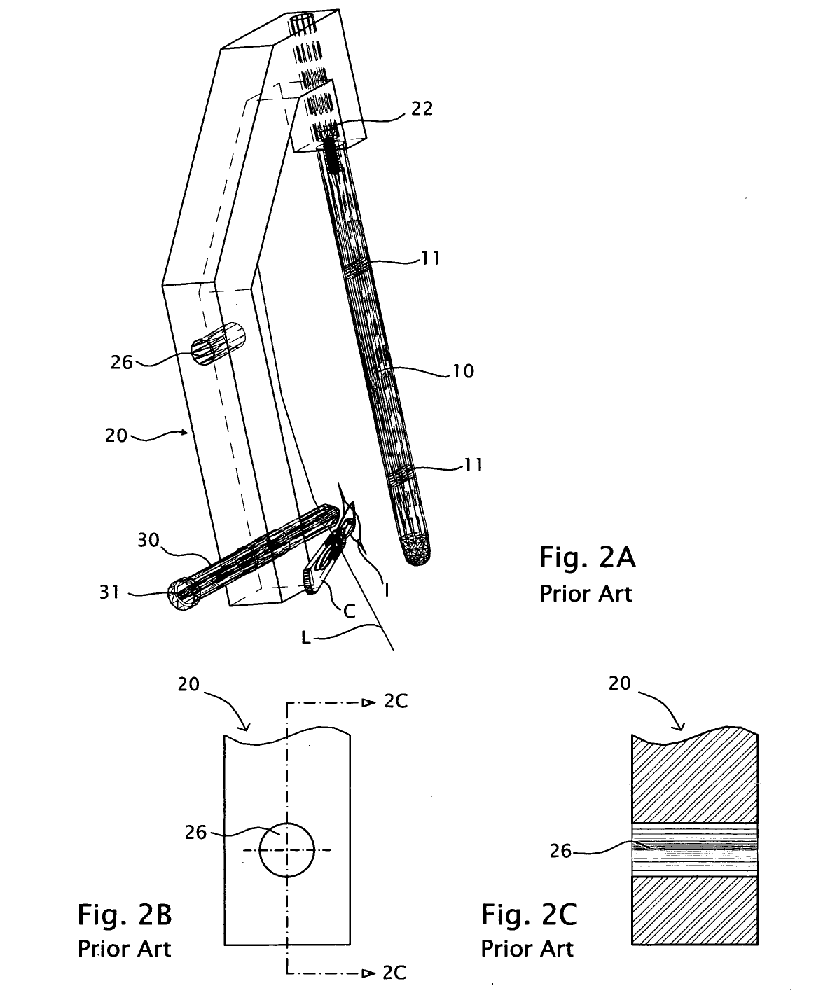

### DETAILED DESCRIPTION OF THE INVENTION

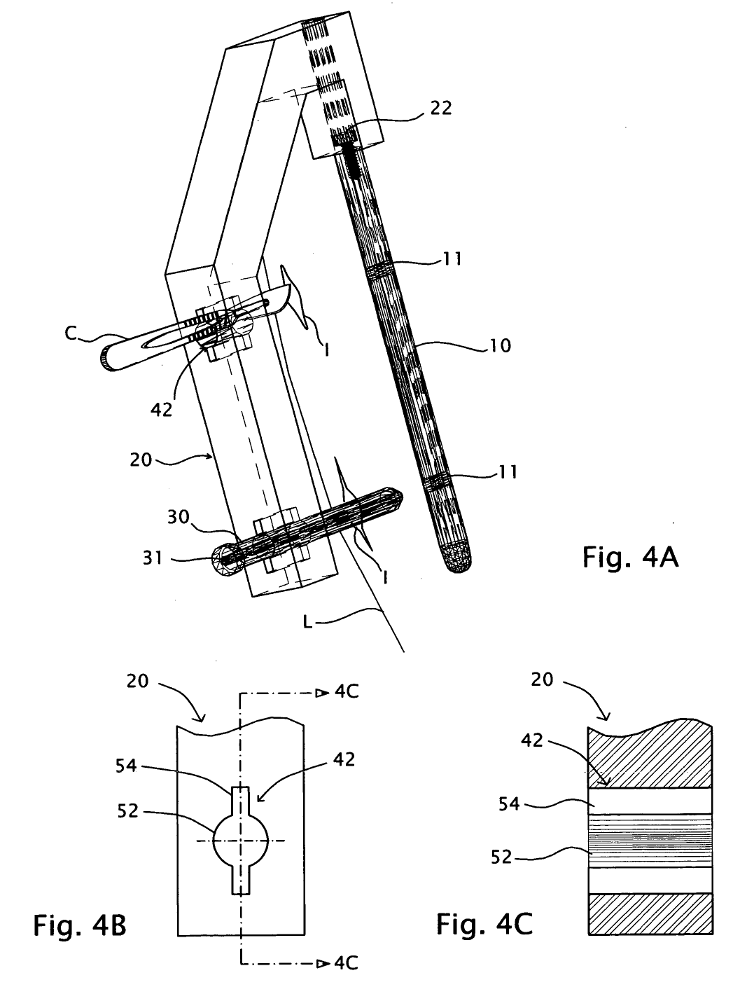

Referring to FIG. 4A there is shown an aiming device 20, which is provided with two transverse holes 42. A locking nail 10 is fixed to the aiming device 20 by means of a screw 22. Locking nail 10 is provided with two transverse holes 11 for receiving appropriate fixation screws (not shown) for anchoring the locking nail 10 in place. This type of fixation operates to prevent the portions of the bone B on either side of the fracture site from moving relative to each other.

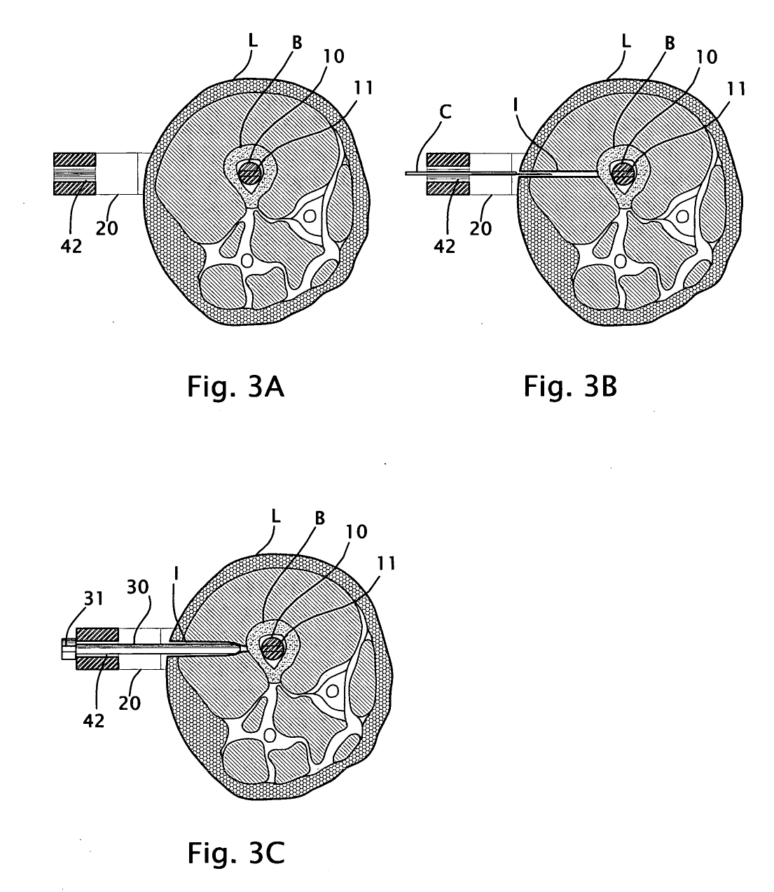

As shown in FIG. 3A the aiming device 20 is correctly positioned so that the aiming device transverse hole 42 is aligned with the nail hole 11.

As shown in FIG. 3B the aiming device 20 transverse hole 42 designed enables the surgeon to insert a cutting element C through the aiming device 20 in order to perform an incision I in a plane that contains nail hole 11 axis.

As shown in FIG. 3C, the drill guide 30 is inserted through the aiming device transverse hole 42 in precise alignment with the nail hole 11 axis. After the drill guide 30 is positioned, a drill (not shown) is inserted through the guide hole 31 so that a hole can be drilled in the bone B. Since the drill guide 30 is aligned with the nail hole 11 and there is no asymmetrical pull stress 18, the drill (not shown) will fit through the hole 11 in the nail 10 and at least one screw (not shown) for anchoring the nail 10 with surrounding bone B can accurately be installed.

As can be recognized from FIG. 4A in the preferred embodiment, aiming device transverse hole 42 comprises a round hole 52 combined with a slot 54. As it can be seen this hole shape allows a cutting instrument C to be inserted through the hole 42 (see FIG. 4B and 4C) in order to perform an incision I.

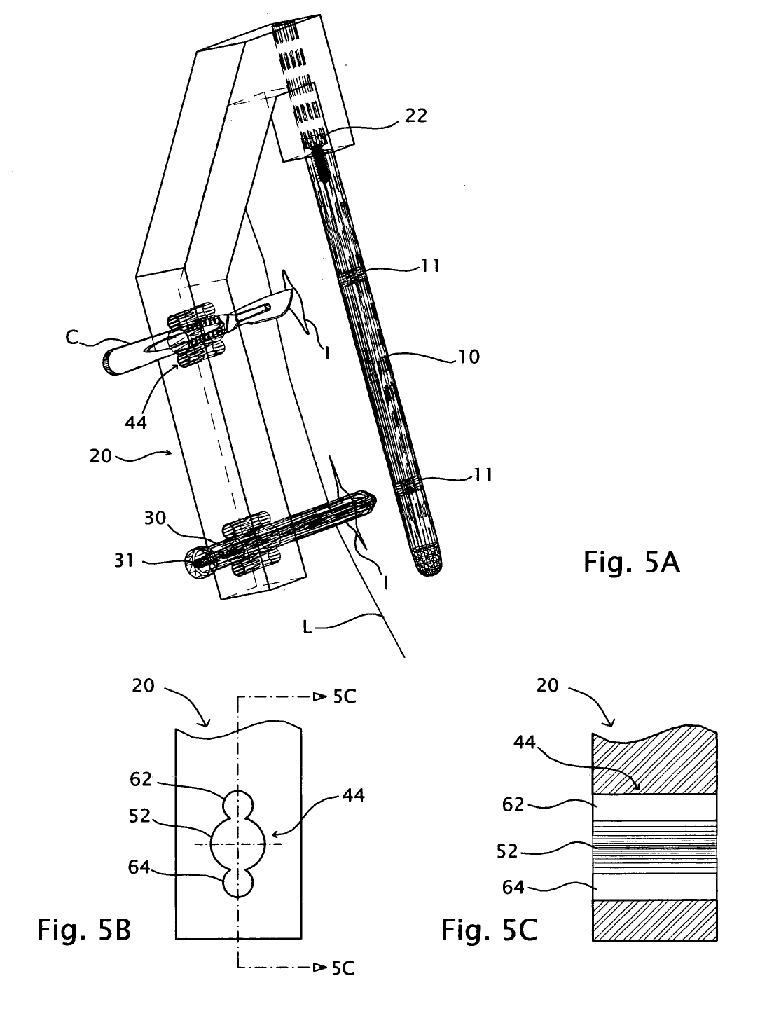

An alternate embodiment is shown in FIG. 5A in which adjacent additional round holes 62, 64 are included. In the aiming device, bores 62, 64 are located so closely to the central round bore 52, that the wall of bores 62, 52 and 64, 52 intersect and are open in the adjoining region (see FIG. 5B and FIG. 5C). Thus, a cutting instrument C can be inserted through the aiming device 20.

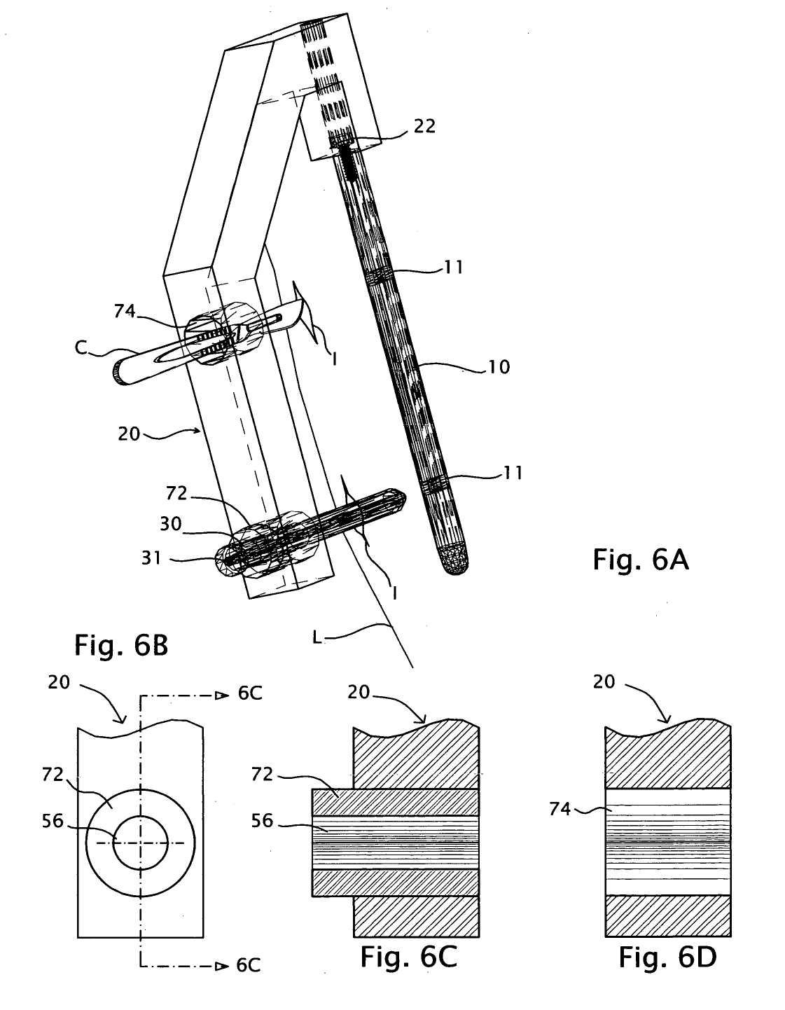

According to a further embodiment of the invention (see FIG. 6A), aiming device transverse hole 56 is placed in a removable insert 72 (see FIG. 6B). Once the insert 72 is removed (see FIG. 6C), a larger hole 74 is left and allows a cutting instrument C to be inserted through the aiming device 20 so that an incision I can be made.

What is claimed is:

1. A special shaped hole located in the aiming device, wherein the special shaped hole comprises a round hole combined with a slot in order to enable the scalpel to go through it.

2. The special shaped hole according to claim 1 wherein the round hole is combined with one or more additional round holes.

3. The special shaped hole according to claim 1 wherein an insert with a transverse hole is fixed in order to allow the drill guide to go through it and when removed enables the scalpel to go through the aiming device.

4. A method to prevent asymmetrical pull stress over the drill guides inserted through the aiming device transverse holes, comprising the steps of:
a) Providing a special shaped hole comprising a round hole combined with a slot.
b) Inserting a scalpel through the special shaped hole so as to perform an incision.
c) Inserting a drill guide sliding through soft tissues without being submitted to asymmetrical pull stress, until external surface of the bone is reached.
d) Inserting an anchoring screw which will perfectly fit through the hole in the nail.

5. The method according to claim 4 wherein the special shaped hole comprises a round hole combined with one or more additional round holes.

6. The method according to claim 4 wherein the special shaped hole comprises an insert with a round hole which enables the drill guide to go through it, and is removed so as to enable the scalpel to go through the aiming device.
